(19) United States
(12) Patent Application Publication
(10) Pub. No.: US 2022/0165393 A1
(43) Pub. Date: May 26, 2022

(54) SYSTEM FOR THE DETECTION AND MANAGEMENT OF MENTAL, EMOTIONAL, AND BEHAVIORAL DISORDERS

(71) Applicant: Inz Human Engineering LLC, Beverly, MA (US)

(72) Inventors: Jonathan Inz, Bedford, MA (US); Matthias A. Pollach, Munich (DE)

(21) Appl. No.: 17/532,836

(22) Filed: Nov. 22, 2021

Related U.S. Application Data
(60) Provisional application No. 63/117,868, filed on Nov. 24, 2020.

(51) Int. Cl. G16H 20/70; G16H 40/67; G16H 80/00; G16H 50/20

(52) U.S. Cl. CPC G16H 20/70; G16H 50/20; G16H 80/00; G16H 40/67

(57) ABSTRACT

A system for the detection and management of behavioral disorders, including but not limited to anxiety and panic attacks, is disclosed. The system incorporates wearable body sensors that measure physiological and behavioral variables, an algorithm for detection of panic attack and other behavioral disorders based on the physiological variables are measured and paired with an application module that can display said variables and detected behavioral disorders to the user, an application module that can instruct a user in real-time management techniques including biofeedback and neurofeedback for a detected disorder, an application module that learns users psychological and physiological patterns for the purpose of self-control and correction. Allows for simultaneous monitoring by health care professionals with real-time intervention.

### CROSS-REFERENCE TO RELATED APPLICATION(S)

This application claims priority to, and the benefit of, U.S. Provisional Application 63/117,868, filed Nov. 24, 2020, for all subject matter common to both applications. The disclosure of said provisional application is hereby incorporated by reference in its entirety.

### FIELD OF THE INVENTION

The present invention relates to a system for detecting and managing various types of mental, emotional, and behavioral disorders. More specifically, the present invention relates to a system that measures a set of physiological variables over time and presents signals related to those variables to the user who uses those signals to control or eliminate the disorder. In particular, the signal presented to the user is the indication of a developing psychological condition, such as but not limited to a panic attack, and indicates appropriate steps for treatment of the condition. Treatment involves psychological and psychophysiological techniques and concepts that have been clinically proven to successfully treat mental disorders.

### BACKGROUND

Mental, emotional, and behavioral disorders have a very significant prevalence in the US and global populations. The National Institute of Mental Health (NIMH) estimated the prevalence in 2015 of any disorder except substance use disorders at 17.9% for US adults, 21.2% for females, and 14.3% for males. In the US population, anxiety disorders tend to have the highest prevalence among all disorders. Anxiety disorders include generalized anxiety disorder, panic disorder, social anxiety disorder, and specific phobias. According to NIMH, the 12-month prevalence of anxiety disorders for US adults is 18.1% of the population. The lifetime prevalence by age is as follows: 25.1% for 13-18 year olds, 30.2% for 18-29 year olds, 35.1% for 30-44 year olds, 30.8% for 45-59 year olds, and 15.3% for adults over 59 years of age. According to the Anxiety and Depression Association of America, anxiety disorders cost the US more than $42 billion a year (Journal of Clinical Psychiatry 60(7), July 2019), and more than $23 billion of those costs are associated with repeated use of healthcare services. People with anxiety disorders are three to five times more likely to go to their primary care physician and six times more likely to be hospitalized for psychiatric disorders than those who do not suffer from anxiety disorders. In The American Journal of Cardiology (2019), researchers observed that 47 percent of subjects exhibited abnormal anxiety symptoms, which correlated to a high rate of ED recidivism and a low rate of medical diagnoses.

Numerous evidence-based, efficacious programs exist for a wide variety of disorders, but treatment effectiveness for these disorders is limited by the clinician's ability to know the actual physiological stress levels. The present invention serves as an aid to users in treating a wide variety of mental, emotional, and behavioral disorders. By focusing on physiological markers, the system increases the treatment effectiveness and reduces the clinician-dependent variability.

Anxiety is a normal human emotion that everyone has experienced, but anxiety disorders are different. Anxiety disorder is a serious mental illness that interferes with a person's ability to lead a normal life. The diagnosis of anxiety disorder depends on the intensity and duration of symptoms and the degree of dysfunction. There are many different types of treatments for anxiety disorder. Some of the more widely used include medication, psychotherapy, cognitive-behavioral therapy, dietary changes, lifestyle changes, and various biobehavioral treatments such as relaxation therapy, hypnosis, and biofeedback.

The use of physiological monitoring and biofeedback combined have been demonstrated to be a powerful treatment of anxiety disorders.

However, this technology experiences shortcomings. The patient who is becoming anxious is not able to mitigate or abort the attack because the anxiety interferes with their ability to implement treatment. Existing physiological monitoring and biofeedback have not been combined to be comprehensive, coordinated, and predictive in nature to alert the user of a physiological event prior to the user's own conscious awareness and enable the administration of real-time treatment prior to the onset of symptoms, which in turn prevents the development of certain symptoms altogether. With behavioral conditions, such as panic disorders, treatment is often futile after an event has begun.

### SUMMARY

There is a need for a system and method for detecting and proactively managing, mitigating, or even eliminating a variety of mental, emotional, and behavioral disorders. The present invention is directed toward further solutions to address this need, in addition to having other desirable characteristics. Specifically, the present invention provides a system for detecting, managing, and potentially eliminating disorders including but not limited to anxiety and panic attacks. The present invention demonstrates that physiological changes are noticeable in the human body prior to the development of a panic attack or other psychological conditions. The system includes one or more wearable devices worn by the user that measure a set of relevant physiological and behavioral variables, and a signal transformer subsystem that transforms the measured physiological and behavioral data into feedback signals for the user. The system triggers real-time psychological treatments based on personalized estimates of the user's mental and physical states. A therapeutic stimulus is selected by the patient from a library of stimuli and displayed to the user. Thus, by using a predictive algorithm that incorporates numerous combinations of physiological indicators, behavioral measures, and circumstantial measures, treatment is administered promptly and more efficiently than standard treatment techniques after the development of conscious symptoms. Machine learning algorithms are used to detect the psychological state of the user, including dynamic Bayesian networks, neural networks, conditional random fields, hidden Markov models, Kalman filters, fuzzy logic, kernel estimators, clustering algorithms including k-nearest neighbors, learning vector quantization, Gaussian models, and radial basis functions.

The following physiological indicators and physiological measures can be used in various combinations to detect the onset of a panic attack or other behavioral disorders: heart rate; heart rate variability (HRV); electrocardiogram (ECG); core body temperature; heat flow off the body; respiratory rate; galvanic skin response (GSR); electromyography (EMG); electroencephalography—Fast Fourier transform analysis (EEG-FFT); electrooculogram (EOG); blood pressure; hydration level; muscle pressure; activity level; skin temperature; body position and posture; acceleration; and voice tone. The devices that measure physiological and behavioral variables are typically portable, lightweight, wearable, battery-operated, non-invasive, and easy to use. The physiological signals are sent wirelessly or by wire to the signal transformer subsystem. The signal transformer subsystem can be located on a server, a tablet computer, smartwatch, or a smartphone connected to the internet or within range of the transmitting devices and the status reporting device.

The feedback signals help the user control, lessen, or eliminate the disorder. Based on an algorithm involving a combination of these physiological variables, a panic attack or other behavioral disorder can be detected prior to the user being consciously aware of the symptoms. In a preferred embodiment, the status signals are included in the feedback signals sent to the user. By continuously receiving feedback on the user's physiological status in the form of detection signals, the user is able to detect the onset of the disorder, to observe a connection between one's mental state and the feedback signals, and, with practice and guidance, learn how to control, mitigate, or eliminate the disorder.

The system is compatible with any biofeedback wearable device. The system includes separate algorithms for all combinations of the aforementioned physiological indicators. Thus, the application for administering treatment is compatible with a wide variety of biofeedback wearable devices ranging from a simple heart rate monitor to a device that can measure all of the included measures. The user is able to connect any device that measures some or all of the indicators listed above for disorder detection. The system displays the specific combination of physiological measures to the user or any derivative that combines these measures into a single variable or score. This allows the user to actively monitor and control his or her physiology so as to return the measures to within normal limits.

Physiological changes in the human body are discovered prior to the development of a panic attack or other psychological conditions. Therefore, the algorithm is predictive in nature and alerts the user of a physiological event prior to the user's own conscious awareness. This allows for the administration of real-time treatment prior to the onset of symptoms, which in turn prevents the development of symptoms altogether. With behavioral conditions, such as panic disorders, treatment is often futile after an event has begun. Thus, by using the predictive algorithm that incorporates numerous combinations of physiological indicators, treatment is administered promptly and more efficiently than standard treatment techniques after the development of symptoms. In the case of panic, intercepting and interrupting the anxiety attack allows the individual to remain in a more mindful, present, and conscious state as opposed to the Hypothalamic-Pituitary-Adrenal (HPA) Axis alarm which induces the General Adaptation Syndrome of panic. This response is commonly known as fight-or-flight syndrome. By anticipating the full panic attack, the inventive system can keep the individual from having the alarm response. The panic-specific algorithm based on combinations of the previously mentioned physiological measures is used to achieve this and begin treatment prior to the onset of a fight-or-flight response.

The user begins a tutorial that explains the treatment approach and provides a comprehensive explanation of the process involved. This tutorial is followed by a training module for the relaxation techniques that are used to prevent the onset of a panic attack, anxiety, or other behavioral disorder. Based on answers to a group of assessment questionnaires, the user is guided to one of three relaxation techniques. The user can also alternatively train one or all of the techniques. The techniques used in this system are progressive muscle relaxation (PMR), guided imagery (GI), and diaphragmatic breathing. Progressive muscle relaxation begins with deep breathing also known as diaphragmatic breathing. Specifically, this is a breath that is held for 7 seconds and repeated 2-3 times. The system then instructs the user to tense a specific muscle group for an extended period of time and then relax said muscle group. This is repeated for 16 major muscle groups moving upward from the feet to the head. For the guided imagery technique, the user is instructed to visualize a calming and relaxing location or memory. This can be anything from a pleasant memory or general thoughts that are calming to the user.

Diaphragmatic breathing, or deep breathing, is breathing that is done by contracting the diaphragm, a muscle located horizontally between the thoracic cavity and abdominal cavity. Air enters the lungs. The chest does not rise, and the belly expands during this type of breathing. All techniques involve training to a criterion to ensure the user has mastered the concepts. When done correctly, the user's physiological levels will be lower as he or she enters a more relaxed mental state. The training module determines the effectiveness of the technique used and instructs the user in areas of improvement. Once the user has practiced 1, 2, or all of these techniques and the application has determined that he or she has a mastery understanding by training to a certain criterion, the user is ready to use these techniques as a treatment method. In combination with the predictive algorithm, the user is warned of the onset of a behavioral event and guided through the relaxation technique he or she has self-selected until it is determined that the event has been mitigated or aborted entirely. The relaxation training module also involves the regular practice of these techniques so that the user's skills remain intact. The concept of regular practice of the relaxation response is part of the concept of mastery inculcated in the patient's understanding of what is necessary to control the disorder. This additionally provides the user with a schedule and structure which has proven to be beneficial in the treatment of behavioral disorders. In the case of panic disorders, the user is guided through this treatment that has proven to be effective in a clinical setting. In the signal transformer subsystem, which is typically located on a server, a tablet computer, smartwatch, or a smartphone, the user has access to the training and instructional modules that may be in audio, visual, or other forms. Numerous psychological concepts, including but not limited to degrees of freedom and choice, bandwidth, signal detection theory, baseline, and homeostasis are taught to the user that have shown effectiveness in the treatment of panic disorders, anxiety, or other behavioral disorders.

By combining an early detection warning signal with these treatment techniques, the user is provided additional degrees of freedom to choose healthier paths of behavior including but not limited to diaphragmatic breathing, progressive muscle relaxation, and guided imagery. The user is trained in these techniques to a criterion that demonstrates proficiency. Providing an individual with these techniques and demonstrating that they represent an alternative path to symptoms has proven to be an effective treatment for behavioral conditions in a clinical setting. The simple notion that a condition is within one's control and not inevitable allows an individual to avoid negative symptoms and produce healthier physiological patterns. In addition to the relaxation techniques, the training module will also explain the psychological concept of degrees of freedom and choice to the user until he or she has an in-depth understanding. The essential thrust of the degree of freedom concept is that currently, the patient has no choice but to panic. The present invention provides a skill set with crucial information to allow for alternative health choices. During regularly scheduled training sessions and prior to real-time administration of relaxation for a detected symptomatic event, this concept is reinforced to ensure strong understanding. Other psychological concepts will also be included in the training module.

The user must wear the wearable, biofeedback, device of his or her choosing and connect it to the signal transformer subsystem. At this point, the system is fully operational. It actively notifies the user that a panic attack or other behavioral disorder is developing and begins the self-selected relaxation technique. The user receives a reinforcement of the idea of degrees of freedom, real-time instructions for the technique, and additional training when he or she is not suffering from a panic attack or other symptoms. He or she will train to a criterion that will provide for the ability to initiate the skill-based, bio-behavioral technique when alerted to the danger of impending events.

Although consistent physiological patterns are present when comparing panic attacks or other psychological conditions of different individuals, symptoms can be patient-specific to some degree. There are clear parallels between psychological conditions when comparing individuals, but the precise manifestation has variance on a case-by-case basis. Thus, the system also includes a subsystem to adjust and improve the algorithm given feedback from a specific user through machine learning. The algorithm becomes personalized to a user through user feedback where false positives and negatives for symptoms or psychological events are indicated. Through machine learning, the system adjusts to errors indicated by the user and becomes more customized to an individual. This process is continuous thus allowing the system to adjust not only to an individual when he or she begins to use the device, but it also adjusts to the progression of the condition since treatment will inevitably change the user's physiological response to his or her psychological condition.

The inventive system involves an alarm subsystem where the user's psychiatric caregiver is immediately notified of any detected events. This is not only beneficial for the medical professional overseeing the user's psychiatric care, but also to the individual. This subsystem provides the user with an additional sense of security as he or she knows a psychiatrist or other caregiver is aware of the user's mental state and development of his or her behavioral condition. The method of contacting the user's medical professional can be a page, phone call, email, or another method that is ideal for both the user and the medical professional. The medical professional may also communicate directly with the user if he or she deems that to be an appropriate course of action. This results in more efficient treatment of behavioral conditions.

Also, the inventive system described provides the capacity to influence physiology with stimulation. The capacity to influence physiology, particularly central measures of EEG, allows for manipulation of brain function to produce healthier patterns of brain activity. Transcranial magnetic stimulation (TMS) is low voltage electrical stimulation to a specific region of the brain influencing the capacity of neuronal firing. This stimulation can either increase or decrease neural firing thus when this is applied to a specific region of the brain associated with a condition, such as depression or panic, dysfunctional symptoms can be alleviated. This system incorporates TMS with the detection algorithm to treat brain function preemptively. As an example, this methodology can produce the ability to short circuit depressed affect by stimulating areas of the cortex to produce healthier EEG rhythms.

This inventive system's theoretical underpinnings formulate a logical understanding of the relationship between physiology and psychology previously unexamined. The present inventive system develops physiological profiles for all psychiatric diagnoses to better understand and treat the user's psychopathology. These physiological profiles consist of complex patterns derived from the assessment of physiological variables. This understanding allows for descriptions of a patient's psychopathology using physiological measures such that each diagnostic criterion is identifiable/codifiable with distinctive patterns of physiologic activity. In the present invention, these physiological personality profiles present themselves without needing any self-report or psychological inquiry. This physiologic taxonomy emerges from the monitoring, assessing, analyzing, and patterning of these variables conjoined with the inventive system for understanding these relationships. These profiles are then synthesized into a psychological condition database that is used to more efficiently diagnose and treat these disorders. In the case of panic, profiles of this nature streamline the diagnosis of panic thus reducing unnecessary patient suffering and costly testing.

In accordance with example embodiments of the present invention, a system for detecting and managing behavioral disorders of a user includes one or more computing devices having a signal processing subsystem and a signal presentation subsystem. One or more wearable body sensors measure one or more physiological indicator variables to obtain physiological data comprising heart rate variability data and transmit the physiological data over a communication network to the signal processing subsystem, wherein the signal processing subsystem determines the onset of a panic attack or other psychological disorder of the user based on the physiological indicator variables and the physiological data and derives an algorithm from differences between a baseline and a shift in physiological arousal exceeding predetermined threshold limits. A status reporting device connected to the communication network receives feedback signals from the signal presentation subsystem. An instruction module is connected to the communication network. The signal processing subsystem uses the algorithm and machine learning to perform detection and classification and transmits detection and classification signals to the signal presentation subsystem, and both are connected to the communication network.

In accordance with aspects of the present invention, the signal processing subsystem includes a remote computing device that transmits physiological data over the communication network to the signal presentation subsystem comprising a second remote computing device and the signal presentation subsystem transmits feedback signals to the status reporting device connected to the communication network and the instruction module connected to the communication network. The communication network is the internet. The status reporting device is connected to the communication network via a Wi-Fi network. The system can further include alarm and status signals that are sent to a therapist who communicates remotely with the user.

In accordance with aspects of the present invention, physiological indicators are used in combination to detect an onset of a panic attack or other behavioral disorder or create an algorithm for detection and the physiological indicators comprise two or more of: heart rate; heart rate variability; electrocardiogram (ECG); respiratory rate; galvanic skin response; electromyography (EMG); electrooculography (EOG); electroencephalography Fast Fourier transform analysis (EEG-FFT); skin temperature; posture; and acceleration. Onset indication functions in combination with real-time medical/symptomatic treatment can be provided, wherein the real-time medical/symptomatic treatment comprises psychological concepts of one or more of conditioning and learning, baseline, degrees of freedom, double helix, and a nature vs. nurture continuum independently or in conjunction with progressive muscle relaxation, guided imagery, or other relaxation techniques.

In accordance with aspects of the present invention, the system further includes treatment using psychological concepts of one or more of conditioning and learning, baseline, degrees of freedom, double helix, and a nature vs. nurture continuum independently or in conjunction with progressive muscle relaxation, guided imagery, or other relaxation techniques.

In accordance with aspects of the present invention, the signal processing subsystem includes a machine learning subsystem that uses machine learning to map features representing physiological, behavioral and circumstantial data over time into reliable behavioral disorder detection and classification signals. The machine learning subsystem can include unsupervised learning and supervised learning modes. The machine learning subsystem unsupervised learning mode can include one or more of clustering and autoencoding to learn higher-order features. The machine learning subsystem can include one or more of a support vector machine, a neural network, a statistical learning algorithm, or a combination thereof.

In accordance with aspects of the present invention, the signal processing subsystem can include a preprocessing subsystem, a feature extraction subsystem, a feature enhancement subsystem, and a machine learning subsystem. The preprocessing subsystem normalizes physiological signals, behavioral measures, and circumstantial measures for each time frame by (a) computing a mean-subtracted and standard deviation normalized signal, (b) computing a range normalized signal, producing a signal range from about 0 to 1 or from −1 to 1, or (c) by computing a ratio of the signal to its L1 or L2 norm. The feature extraction subsystem can extract a set of features by determining the closest match of the signal over each time frame to a dictionary of functions and/or a predefined set of functions. The feature extraction subsystem can compute one or more of a Fast Fourier Transform, a wavelet transform, a principal components analysis, an independent components analysis, or a bank of bandpass filters.

In accordance with aspects of the present invention, the system monitors automated feedback and characterizes the automated feedback for effectiveness. Clinician-provided feedback can be monitored and characterized for effectiveness. Machine learning can be used to improve treatment effectiveness based on the most effective automated and clinician-provided feedback. Treatment effectiveness can be estimated using a reduction in the number or severity of behavioral disorders from a baseline.

In accordance with aspects of the present invention, data are collected from multiple users and used to establish the baseline and a baseline physiological profile for each user. Data can be collected from multiple users and used to establish the baseline and a baseline physiological profile for a group of users. The baseline and the baseline physiological profile can be based on classification and detection signals. The baseline can include heart rate variability data values derived from clinical data sets or aggregated data sets collected from multiple users.

In accordance with aspects of the present invention, the processing subsystem can use machine learning to adjust an algorithm according to physiological indicators that comprise two or more of heart rate (HR), heart rate variability (HRV), electrocardiogram (ECG), respiratory rate (RR), galvanic skin response (GSR), electromyography (EMG), electrooculography (EOG), electroencephalography Fast Fourier transform analysis (EEG-FFT), skin temperature, posture, and acceleration based feedback of the user or specific physiology of the user in order to modify treatment.

In accordance with aspects of the present invention, the system instructs the user in real-time, using the instruction module, with management techniques comprising biofeedback and/or neurofeedback for a detected psychological disorder, wherein feedback signals and status signals are sent to the user via the status reporting device comprising one of a smartwatch, a smartphone, a tablet computer, a laptop, or personal computing device presenting one or more of visual, audio, textual, vibrational or kinesthetic signals to the user.

In accordance with aspects of the present invention, the system further includes the processing subsystem using a machine learning subsystem to identify user psychological patterns and/or physiological patterns demonstrating characteristics of psychiatric presentations that comprise physiological changes, enabling a physiological signal to a set criterion specific to the user, triggering an alert signal that will be provided to the user when a next disorder event takes place based on user psychological patterns and/or physiological patterns.

In accordance with aspects of the present invention, the behavioral disorders of the user include one or more of panic disorder, anxiety, or depression of the user.

In accordance with aspects of the present invention, the one or more wearable body sensors can include electrodes or charge and/or voltage measuring devices configured to record ECG data from the user.

In accordance with aspects of the present invention, the baseline can include heart rate variability data values determined using a machine learning subsystem to analyze prior recorded heart rate variability of one or more users previously transmitted by one or more wearable body sensors attached to the one or more users.

In accordance with embodiments of the present invention, a system for detecting and managing behavioral disorders of a user includes one or more wearable body sensors that measure one or more physiological indicator variables comprising heart rate variability to obtain physiological data and transmit physiological data to a signal processing subsystem comprising a computing device. The signal processing subsystem can determine the onset of a panic attack or other psychological disorder based on the physiological indicator variables and the physiological data to derive an algorithm from differences between a baseline and a shift in physiological arousal exceeding predetermined threshold limits. A signal presentation subsystem can include a computing device that electronically transmits feedback signals to a status reporting device and an instruction module. The signal processing subsystem can use the algorithm and machine learning to perform detection and classification, and transmits detection and classification signals to the signal presentation subsystem, and the signal processing subsystem, the signal presentation subsystem, the instruction module, and the status reporting device are in electronic communication.

In accordance with aspects of the present invention, the signal processing subsystem and the signal presentation subsystem can both be located on the status reporting device and no connection to an external communication network is required.

In accordance with embodiments of the present invention, a method for detecting and managing behavioral disorders of a user includes one or more wearable body sensors measuring one or more physiological indicator variables comprising heart rate variability to obtain physiological data from the user wearing the one or more wearable body sensors. The one or more wearable body sensors can wirelessly transmit the physiological data over a communication network to a signal processing subsystem comprising a remote computing device. The signal processing subsystem can determine the onset of a panic attack or other psychological disorder based on the physiological indicator variables and the physiological data and deriving an algorithm from differences between a baseline and a shift in physiological arousal exceeding predetermined threshold limits. The signal processing subsystem can perform detection and classification using the algorithm and machine learning, and transmitting detection and classification signals to a signal presentation subsystem, comprising the remote computing device, that transmits feedback signals to a status reporting device presenting feedback signals and status signals to the user. An instruction module can present to the user appropriate steps for the management and treatment of a detected psychological disorder. The signal processing subsystem, the signal presentation subsystem, the instruction module, and the status reporting device can be connected to the communication network.

### BRIEF DESCRIPTION OF THE FIGURES

These and other characteristics of the present invention will be more fully understood by reference to the following detailed description in conjunction with the attached drawings, in which:

FIG. 1 is a diagram of the detection and management of mental, emotional, and behavioral systems;

FIG. 2 is a block diagram of a state transition and state prediction model;

FIG. 3 is a block diagram of Panic States measured by arousal level;

FIG. 4 is a block diagram of an Evolving Algorithm;

FIG. 5 is a block diagram of a Signal Transformer Subsystem;

FIG. 6 is a block diagram of a Signal Processing Subsystem;

FIG. 7 is a block diagram of a Preprocessing Subsystem;

FIG. 8 is a block diagram of a Feature Extraction Subsystem;

FIG. 9 is a block diagram of a Machine Learning Subsystem;

FIG. 10 is a block diagram of input and output using neurons nodes;

FIG. 11 is a block diagram of weighting for activation function; and

FIG. 12 is a flow chart indicating signal flow from the body sensors on the user to the signal processing subsystem to decision points to the signal presentation subsystem to the therapist, and to the user.

### DETAILED DESCRIPTION

### Glossary

The purpose of this glossary is to provide a general definition of some terms for which the meaning may not be obvious to the reader. These are common definitions that may be found in a variety of places, including Wikipedia.

Autoencoder—a feedforward neural network in which the output neural layer is identical to the input neural layer.

Backpropagation—a training method for a feedforward neural network.

Bidirectional associative memory—a hetero-associative neural network with two neural layers. Once trained, the presentation of a pattern in one layer will return the associatively learned pattern in the other layer.

Deep learning neural network—a neural network including two or more hidden layers of neurons.

Feedforward neural network—a neural network including an input neural layer connected to one or more hidden neural layers, and an output neural layer.

Independent components analysis—a computational method for separating a signal into additive, statistically independent subcomponents.

L1 norm—the sum of the absolute values of a set of values.

L2 norm—the square root of the sum of the squares of a set of values.

Matching pursuit—a computational method that projects multidimensional data onto a subset of functions of a dictionary of functions.

Neural layer—a set of neurons.

Neural network—a set of neural layers connected via adjustable parameters or weights.

Principal components analysis—a statistical method consisting of an orthogonal transformation that projects multidimensional data onto a set of linearly uncorrelated variables, called principal components, such that the components are ordered with respect to their variance.

Supervised learning—a learning method in which a neural network is provided with pairs of input vectors and output vectors. The output vectors can be thought of as labels that the neural network learns to associate with the input vectors.

Support vector machine—a machine learning method that constructs a set of hyperplanes that can be used for classification, regression, or detection of multidimensional data.

Unsupervised learning—a learning method in which a neural network is provided with input vectors and the network builds internal representations of the input vectors. In contrast with supervised learning, unsupervised learning does not involve a set of desired output vectors or labels.

An illustrative embodiment of the present invention relates to a system and method for the detection and management of behavioral disorders that incorporates wearable body sensors that measure physiological and behavioral variables, algorithms for detection of panic attack and other behavioral disorders based on measured physiological variables, and an application module that displays said variables and detected behavioral disorders to a user and instructs a user in real-time with management techniques including biofeedback and neurofeedback for a detected disorder.

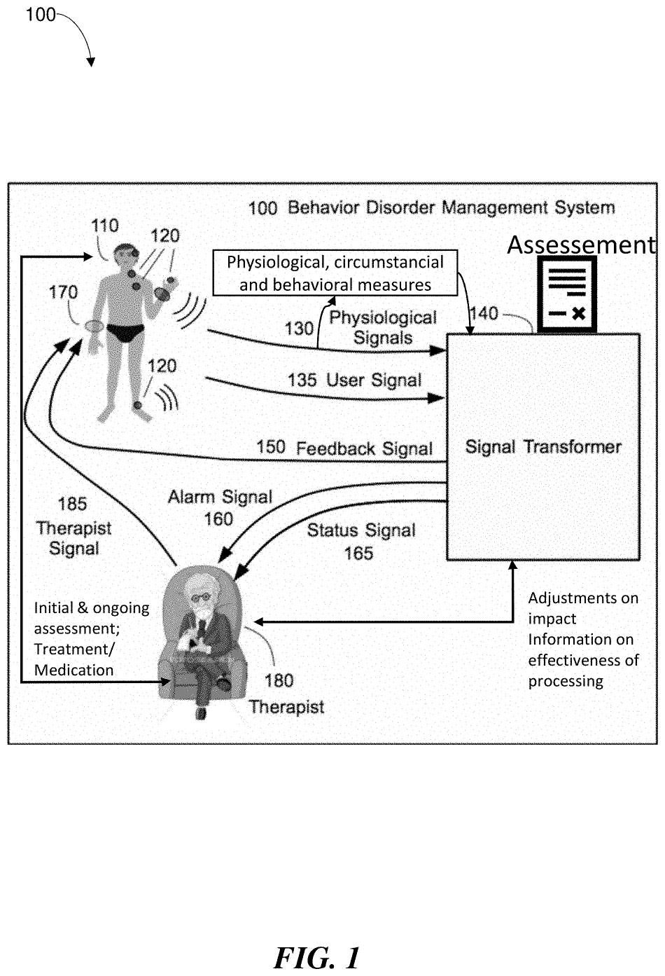

FIGS. 1 through 12, wherein like parts are designated by like reference numerals throughout, illustrate an example embodiment or embodiments of a system for the detection and management of behavioral disorders based on wearable body sensors that measure physiological and behavioral variables that provides biofeedback and neurofeedback using an application module, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, in a manner still in keeping with the spirit and scope of the present invention.

Particular reference is now made to the figures, where it is understood that like reference numbers refer to like elements. A behavior disorder management system in a configuration according to the concepts of the present invention is designated by the numeral 100 in FIG. 1 of the drawings. The system 100 comprises various body sensors 120 located on the body of the user 110. The various body sensors 120 may include, but are not limited to, sensors for measuring heart (electrocardiogram) signals and heart rate, respiration rate, skin temperature, skin conductance, scalp electrical activity, muscle electrical activity, body position, and movement. The body sensors 120 transmit physiological signals 130 wirelessly or by wire to a signal transformer 140. The body sensors 120 may be disposed within or connected to a user 110 device that includes transceivers or transmission components known in the art and may be included in the status reporting device 170 used to report data from the system 100 to the user 110. Physiological signals 130 communicate various physiological measures collected by the body sensors 120. The physiological measures and signals may include, but are not limited to, movement data via e.g., internal IMU in smartphone/device; ECG or similar unprocessed/raw signal; Heart Rate Variability (HRV) or processed ECG signal used to extract Heart Rate Variability (HRV); measures based upon or derived from engineered features—e.g., gradients, maxima/minima, dynamic range, fast Fourier transformed signals, spectrogram, energy/amplitude of ECG. Additionally, several measures correspond to engineered features based on oxygen level including, but not limited to: Galvanic skin response; Oxygen level; Eye blinking; EEG/brain activity. The system 100 also measures changes of changes on any of the above signals when applying when of the therapy methods offered by Zoe. Physiological signals 130 may also correspond to measurements of Hormonal state. The body sensors 120 and corresponding user 110 devices 170 may also transmit or provide signals describing circumstantial 136 measures.

Additionally, electrodes may be added to allow for stimulation of the brain using low voltage signals for the purpose of neuromodulation of brain wave activity. This process, known as Transcranial Magnetic Stimulation (TMS) is capable of positively influencing brain function to decrease psychiatric and dysfunctional patterns of this activity

Circumstantial measures include, but are not limited to: a number of people in the immediate surroundings of a user 110 (measured by unique device signals that can be captured e.g. by device pings or handshakes or service data within sensor or device range); Indoor or Outdoor location (based on displacements, direction changes, and velocities recorded and/or transmitted); proximity to point of interests (POI) —bridges, work, home etc. (derived from e.g. online or electronic maps, GPS signal or known home and/or work addresses or coordinates); travel (e.g. GPS position movement); movements patterns (e.g. increased activity or repeated activity in close proximity to home); news, weather, storm warnings, or calendar information (annotated as to certain categories); and time (e.g. day, night or specified period). The body sensors 120 and corresponding user 110 devices 170 may also transmit or provide signals describing behavioral 137 measures. Behavioral measures include, but are not limited to: movement patterns; food choices (e.g. input via smartphone or hunger determined by lack of input or cross-referenced physiological signals 130); screen-time on smartphone, usage of certain applications (“apps”) or response to alerts on the smartphones or similar user 110 devices 170; Interaction with Zoe application (e.g. therapy using one of the offered techniques is started, relaxation method is started, viewing feedback from app or listened to feedback from app etc.); recorded reaction to selected treatment method implementation; and (sleep activity measured by user 110 device to determine if a person is tired during a specified interval).

The signal transformer 140 processes the physiological signals 130 and transforms them into one or more of visual, audio, textual, vibrational, or kinesthetic feedback signals 150 for the user 110 as well as alarm 160 and status 165 signals for a therapist 180. The status signals 165 are included in the feedback signals sent to the user 110. The feedback 150 and status signals 165 are sent to the user 110 via a status reporting device 170 that is worn or held by the user 110. The status reporting device 170 can be a smartwatch, a smartphone, a tablet computer, a laptop, or any other computing and communicating device that is capable of presenting visual, audio, textual, vibrational, or kinesthetic signals to the user 110. The status reporting device 170 also enables the user 110 to generate signals 135 to mark an event class and severity and to annotate the event with text and/or a voice recording.

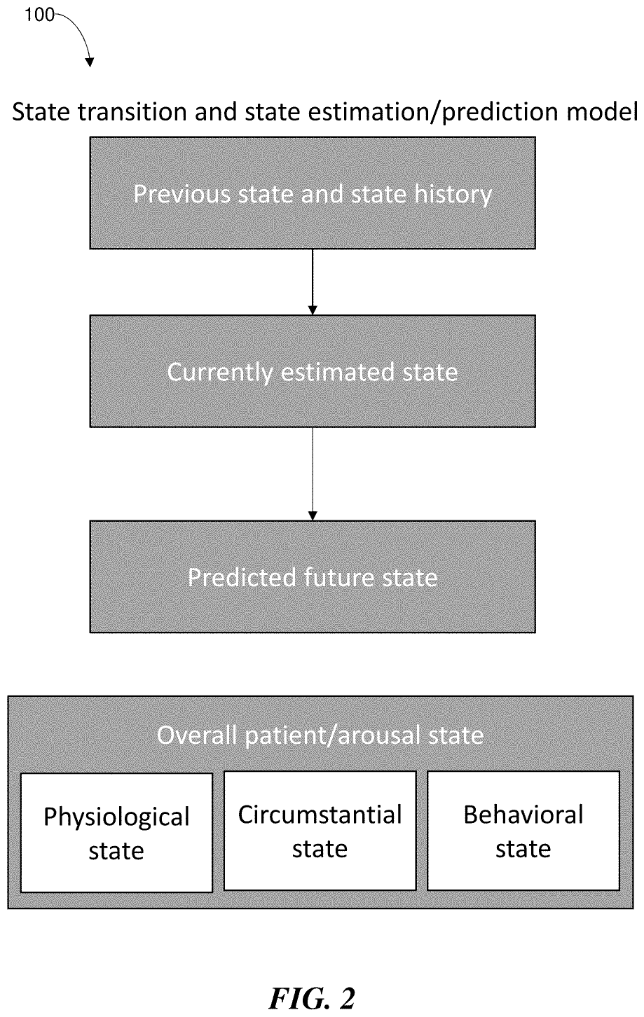

Additional control of the system 100 and data processed by the system 100 and/or provided to the status reporting device 170 may take the form of therapist input entered by a separate computing device with e.g., wireless telecommunication and networking capability used by a therapist 180 of the relevant user 110 or patient. Therapist input may include an initial patient assessment derived from a questionnaire or otherwise assessing how a particular patient reacts in certain situations such as the reaction when interacting with other people (e.g., patient feels uncomfortable when other people are around). The initial assessment may also include ascertaining how a patient experiences panic, whether it be e.g., strong movements, no movement (e.g., frozen) or some other manifestation. After the initial assessment, additional therapist input may include ongoing patient monitoring, where feedback delivered by a therapist 180 may evaluate the effectiveness of certain treatment methods or provide adjustment based on measured impact severity of any of the other signals, changing treatment protocols and signals accordingly. FIG. 2 depicts a block diagram of the state transition and state estimation and/or prediction model used to process incoming signal data from a patient (including physiological 130, circumstantial 136, and behavioral 137 measures) that is used by the system 100 in detecting, evaluating and managing various types of mental, emotional, and behavioral disorders through the operation of the system 100 including treatments, therapies, notifications, alarms, and interventions delivered to the patient by the system 100. The system 100 processes incoming physiological, circumstantial, and behavioral data signals 130, 136, 137 in conjunction with existing system 100 software and logic including data stores of disorder and patient information and combines the various data to establish a current state of the patient being evaluated by the system 100 including an overall patient arousal state derived from the real-time transmission of current physiological, circumstantial and behavioral data signals 130, 136, 137 that indicate, inter alia, a patient physiological state, circumstantial state, and behavioral state that is recorded by the system 100, saved, and used in further processing. Data recorded by the system 100 and saved in a data store (e.g., local or cloud-based database or blockchain distributed storage) is used with the state transition and state estimation and/or prediction model to provide a previous state and state history of the particular patient. Data is continually processed from incoming signals to establish a currently estimated state for the user 110, and model data is used to parse signal data, apply model parameters, and evaluate derived measures to establish a predicted future state for that patient, that is then used to assess if the system 100 needs to take further steps interacting with that patient by e.g. treatments, therapies, notifications, alarms and interventions delivered to the patient by the system 100 (which are in turn continually monitored in an ongoing iterative process).

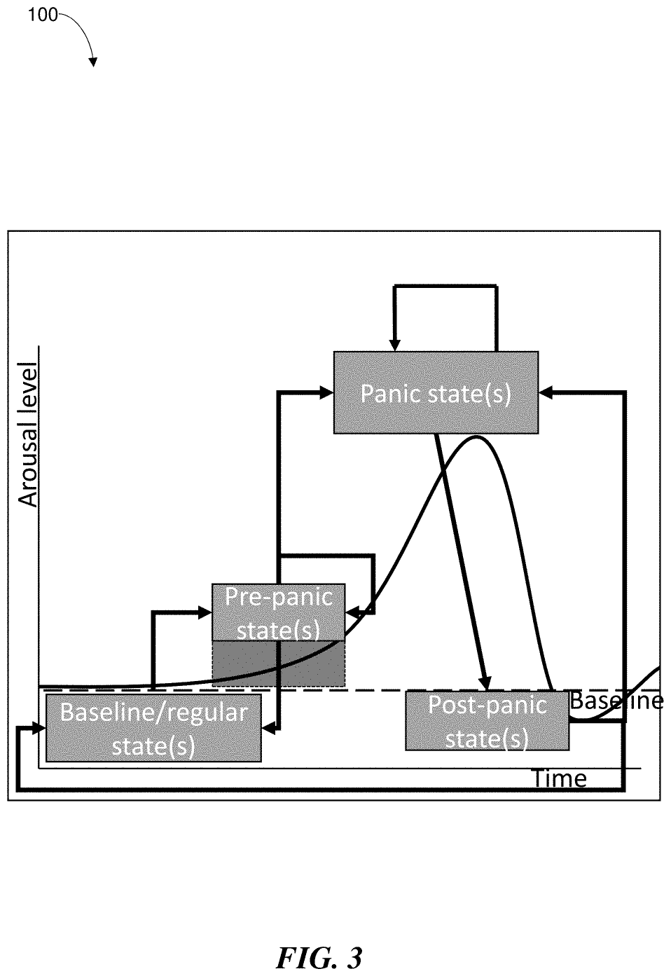

FIG. 3 depicts a block diagram of an example embodiment of common progressions of patient states that are monitored, evaluated, and managed by the system 100. FIG. 3 depicts these states plotted according to arousal level overall patient/arousal state) over time. In the graph of time plotted against a dependent variable of arousal state, a patient begins at a baseline/regular state with an arousal level (measured by received signal data including e.g., heart rate and HRV) that falls within established norms and thresholds for arousal. Over time, patient activities or external stimuli cause the patient to begin to transition to other states. Patient arousal increases and the patient progresses to a pre-panic state prior to panic where intervention by the system 100 through treatments, alarms, etc. may be employed prior to the onset of a panic state such that the subsequent panic may be avoided. Without adjustment, arousal level then rises dramatically into a panic state (state of panic) that is detrimental to the patient and must be addressed, including by progressing the patient to a post-panic state that corresponds to a period where a significant decline in arousal level is experienced. The process may then return to a baseline for the patient and may repeat iteratively as the patient state changes over time. Note, however, that these stages of the progression are not the only path, where for example, a patient may experience a return to the pre-panic state from a panic state or a return to a baseline/regular state from a pre-panic state, as well as other transitions. States measured by arousal level are evaluated and updated in real-time to accurately provide assistance to the patient. Experienced states are also logged and recorded in the data store to become previous states in the state history of the patient.

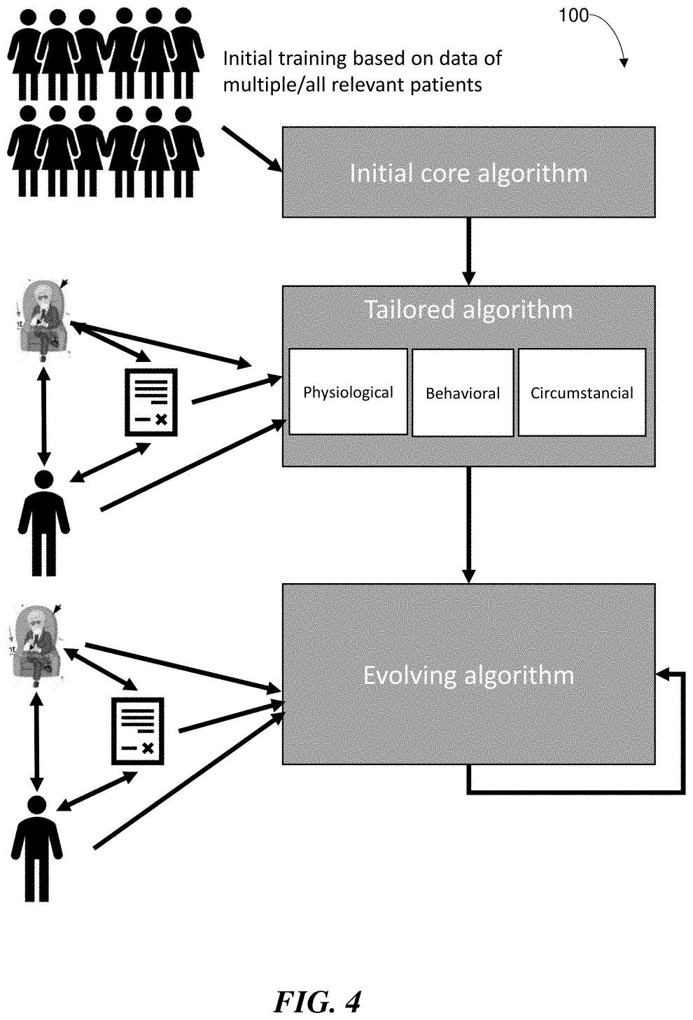

FIG. 4 depicts a block diagram of an example embodiment of how system 100 algorithms that return output based on sets of input from patients 110 and therapists 180 are developed over time to better assist users 110 and patients in detecting, evaluating, and managing various types of mental, emotional, and behavioral disorders. An initial core algorithm for responding to patient signal 130 input is developed and trained from a population of patients using data collected from all relevant patients or multiple patients in a subset thereof. Next, the algorithm is tailored and customized to a specific application for which the system 100 is prepared, most often a specific patient. Tailoring includes processing particular instances of patient physiological state, circumstantial state, behavioral state data collected from that patient and/or patient devices 170 to respond to individual characteristics. Tailoring also includes processing assessments and other feedback or therapist input to develop patient state data, baselines, alarm levels or thresholds, and output parameters closely linked to the ongoing experience of the patient. The system 100 then continually updates and refines the algorithm through machine learning and processing additional input including patient state and therapist 180 data over time, creating an evolving algorithm with additional data over time that is procured repeatedly and continually from therapist input or progressing data collection from the patient after administration of various treatment protocols. Thus, as the patient states are adjusted over time, causing the patient to exhibit altered state data (e.g., the patient responds positively to system 100 administered treatment and experiences fewer symptoms or altered transitions in arousal levels as a result) the algorithm also continues to be refined with the new patient data to progress along with the patient.

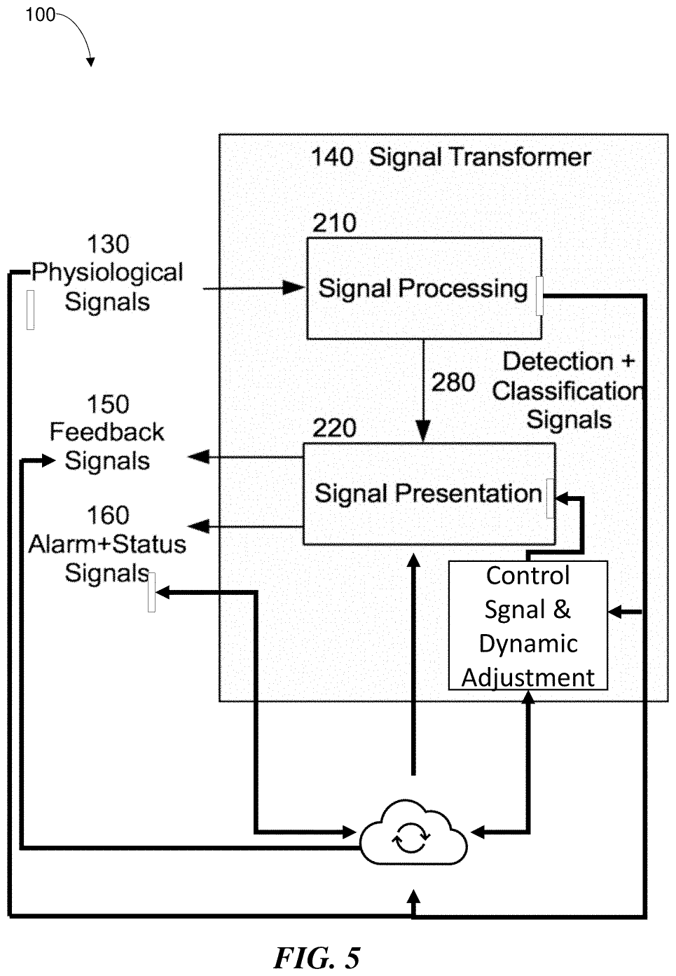

A block diagram of the signal transformer subsystem 140 is provided in FIG. 5 of the drawings. The signal transformer 140 (equivalent to 140 in FIG. 1) comprises a signal processing subsystem 210 and a signal presentation subsystem 220. The signal processing subsystem 210 accepts physiological signals 130 as well as circumstantial measure signals and behavioral measure signals from the body sensors 120 and converts them into detection and classification signals 280 that are used by the signal presentation subsystem 220. The signal transformer 140 stores physiological data, circumstantial data, behavioral data, detection signals, and classification signals. The detection signals indicate the degree to which one or more disorders are significantly affecting the user 110 and the classification signals indicate activity of disorder state categories. The classes of states depend on the type of disorder, but for most disorders, the states can be categorized into several classes according to a severity scale. For example, the severity scales may be classified as normal (no disorder), mild, moderate, and intense. In a preferred embodiment, therapist 180 will be sent alarm 160 and/or status 165 signal if one or more of the alarms is activated. Upon receiving an alarm signal 160, the therapist 180 will attempt to contact the user 110 and provide advice 185 for controlling, lessening, or eliminating the disorder. The system 100 provides a multi-stage approach to detecting panic. In one example embodiment, a first stage pre-panic/detector runs constantly to monitor the patient for events in real-time. In case of a potential pre-panic/panic event, the system then implements a more sophisticated classifier in e.g., a cloud resource that is used to evaluate either the raw signal, the pre-processed signal, or both. Adjustments may be performed on the fly and received from therapist 180 and/or user 110/patient. Additional control signals may be used by the system 100 (transmitted and received by system 100 devices and components) to enable further dynamic adjustments to be made to the algorithms and output sent to the user 110/patient.

In an example embodiment, the therapist 180 can be a single therapist 180 or can be part of a pool of therapists 180 who have agreed to be available for receiving alarm 160 and status signals 165 and for providing guidance to the user 110, with each therapist 180 being available during a predefined period of time. The system 100 coordinates the transmission and receipt of relevant data based upon predetermined operating conditions and system parameters that may be altered by e.g., the pool of therapists 180.

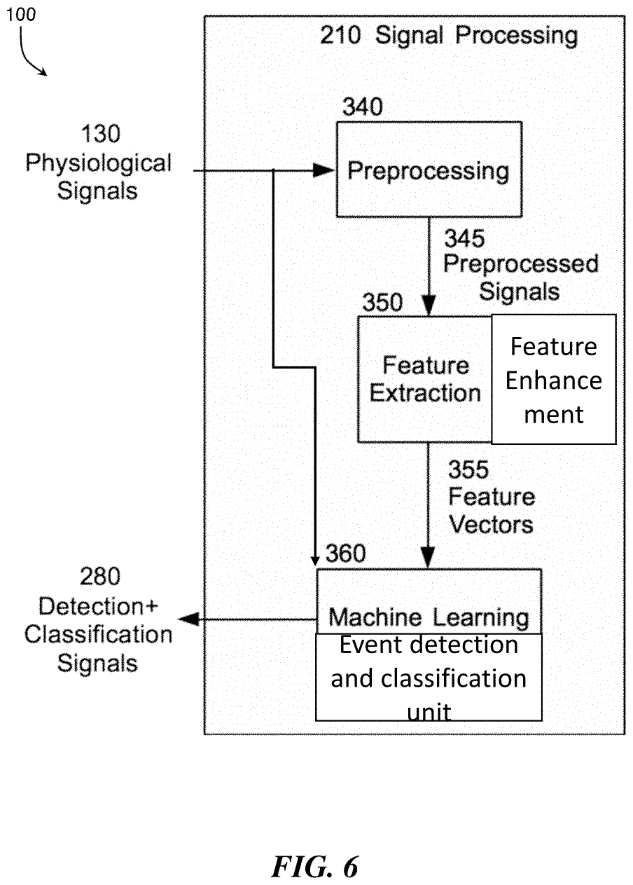

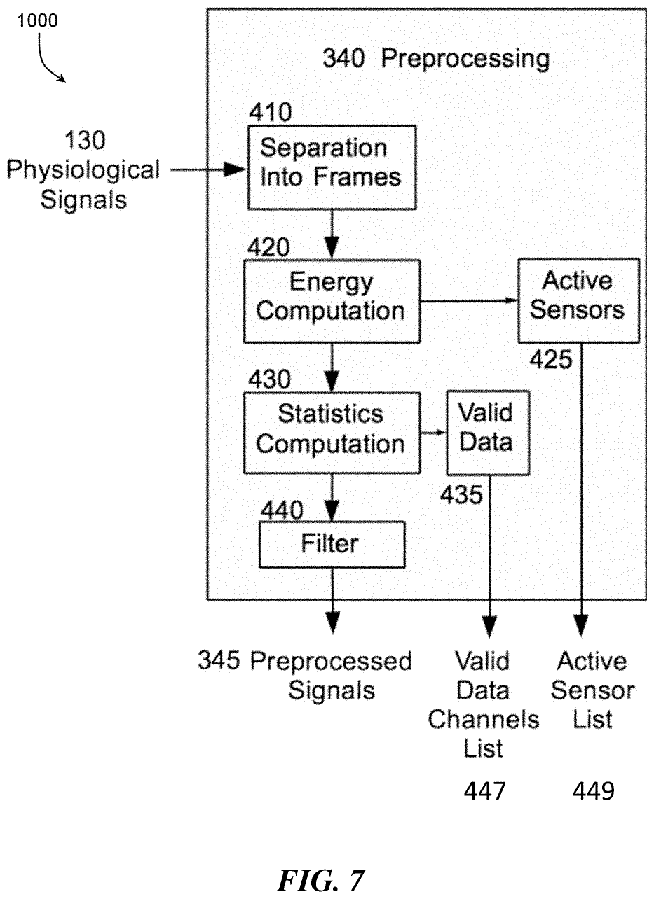

A block diagram of the signal processing subsystem 210 is provided in FIG. 6 of the drawings. The signal processing subsystem 210 (equivalent to 210 in FIG. 5) comprises a preprocessing subsystem 340, a feature extraction subsystem 350, feature enhancement subsystem 352, and a machine learning and refinement subsystem 360. The preprocessing subsystem 340, depicted in FIG. 7, accepts physiological signals 130 from the body sensors as well as signals for circumstantial 136 measures and behavioral 137 measures; separates the signals into time frames 410 that may be disjoint or overlapping; computes the signal energy in each time segment for each physiological signal 130 corresponding to each body sensor 420; compiles a list 449 of active sensors 425, computes various signal statistics including the mean, variance, minimum, and maximum of the signal within each time frame 430; determines which sensor data channels are valid 435 (and aggregates a list 447 thereof) based on energy extrema and other statistics; and filters 440 the physiological signals 130 to provide a set of preprocessed signals 345. Alternative methods of marking time segments as valid or invalid based on other measures are also possible in alternative embodiments. There are multiple ways to denoise and amplify the signal known in the art, including, but not limited to, using classical approaches such as a Wiener filter, etc. In addition, machine learning-based 540 approaches implemented by the system 100 can also be used for denoising, amplification and enrichment (filling in missing signal parts, smoothing, etc.). Other embodiments include multi-rate signal processing.

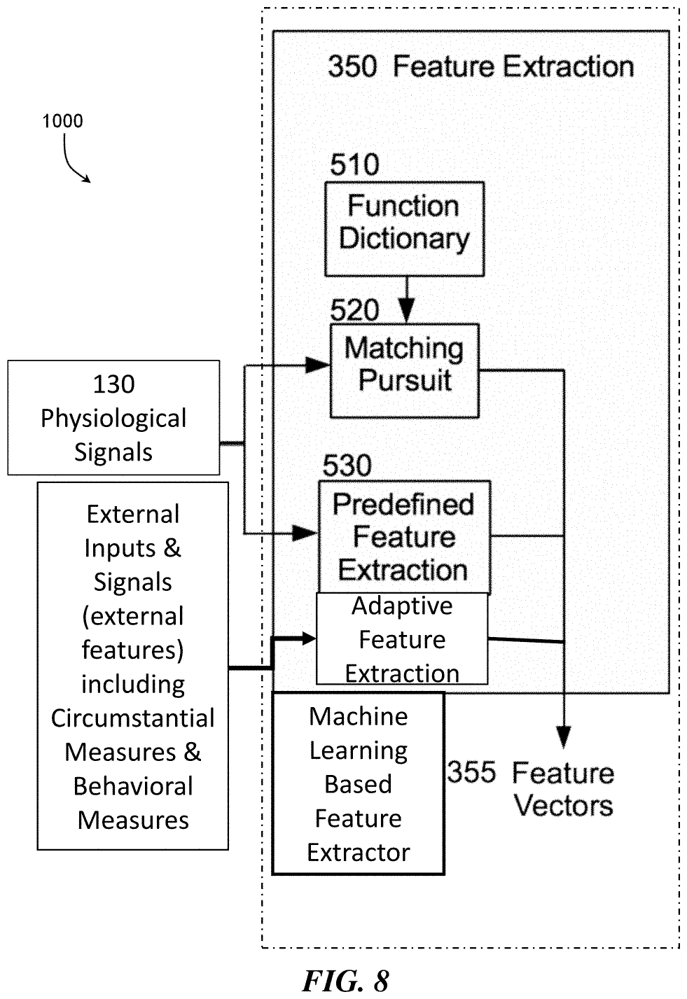

The feature extraction subsystem 350 depicted in FIG. 8 extracts a set of features from the output of the preprocessing subsystem 340. The features may span multiple time frames. The output of the feature extraction subsystem is a set of feature vectors 355 that may consist of a set of functions 510 (or function indices) chosen from a dictionary of functions using a matching method such as matching pursuit 520, a set of predefined features 530 (or predefined feature indices), or both. Additional or alternative features are possible in alternative embodiments. For example, several common signal transforms can also be used to construct features such as Fourier transforms, wavelet transforms, principal components analysis, independent components analysis, or a bank of bandpass filters 440. In an example embodiment, an adaptive feature extraction is also implemented based on external inputs and signals (external features) including signals corresponding to circumstantial 136 measures and behavioral 137 measures. In addition, a machine learning-based feature extractor is also employed to define new features, indices, functions, etc., to capture features uniquely descriptive of individual patients.

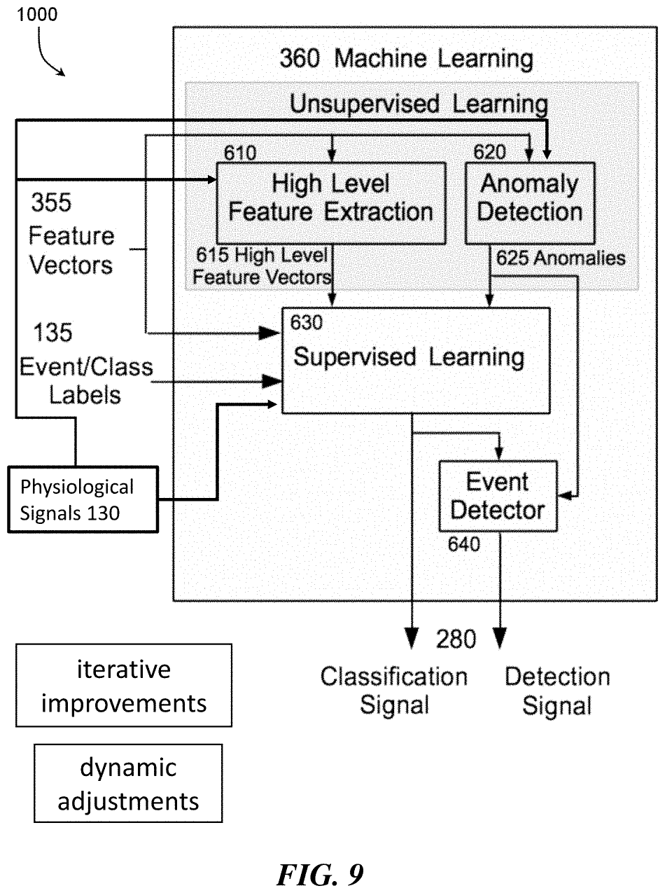

FIG. 9 depicts a block diagram of the machine learning subsystem 360. The machine learning subsystem 360 operates in three modes: an unsupervised learning mode, a supervised learning mode, and a non-learning mode. In the unsupervised learning mode, features are clustered, and this clustering process creates a set of higher-level features. Alternatively, or in addition to the clustering process, an autoencoder is used to learn higher-level features 610 from the feature vectors 615. The autoencoder's hidden units encode higher-level features 610 of the input feature vectors 615. In the unsupervised learning mode, the machine learning subsystem 360 also performs anomaly detection and adapts its adjustable parameters to learn conditions under which anomalies are observed. The sensitivity of the machine learning subsystem 360 to anomalies is used to determine the phase transitions between anomalies and to choose an appropriate number of classes of behaviors. In the supervised learning mode, explicit labels are provided by the user 110, by a therapist 180, or by another party that indicate the mental, emotional, or behavioral status of the user 110. These labels are paired with the features and a mapping from features to labels is formed via associative or supervised learning. An event detector 640 accepts anomaly signals 625 from the anomaly detector 620 and event or classification labels from the supervised learning subsystem 630. In the non-learning mode, the learned parameters (or weights) of the system 100 are fixed. The three learning modes may operate sequentially or in parallel. In a preferred embodiment, the unsupervised and supervised learning modes operate in parallel, and the learned parameters (or weights) of the two learning modes are periodically updated at the status reporting device 170. The machine learning subsystem 360 may employ a support vector machine, a bidirectional associative memory, a feedforward neural network trained using backpropagation, a deep learning neural network, or a combination of all of these. Alternative machine learning systems and options are also possible in alternative embodiments. Note that the iterative process of inference from the data and training using the data may be interoperable and occur iteratively at different or simultaneous intervals. Moreover, the iterative improvements of the system 100 may include dynamic adjustments in addition to other vectors for system 100 evolution.

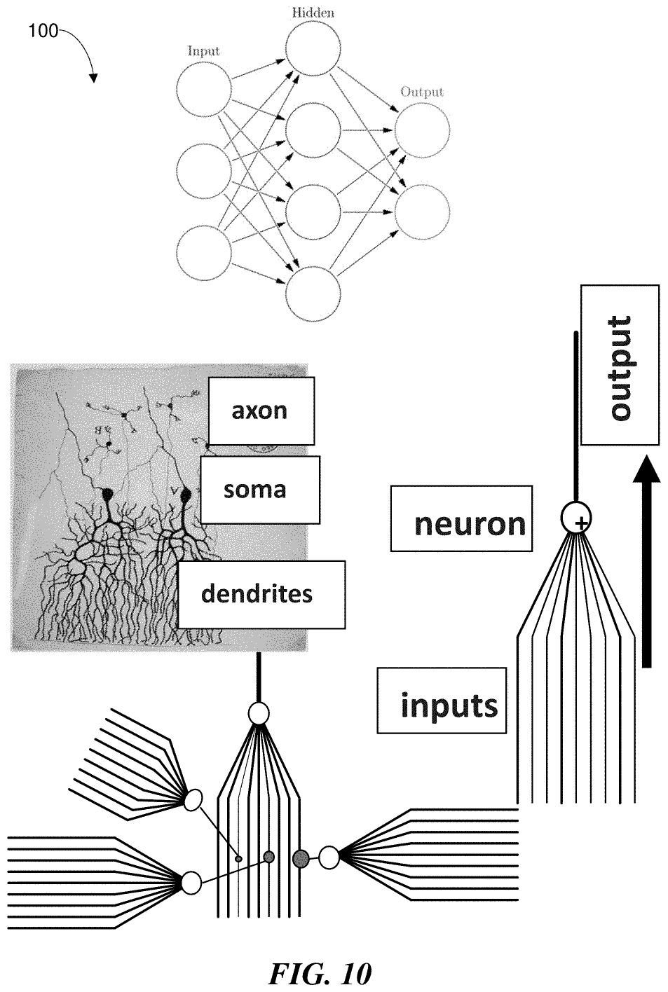

FIG. 10 depicts a block diagram of an example embodiment of input and output using neurons (nodes) connected by weighted paths describing relationships thereof. The machine learning architecture of the system 100 provides input and output values that are labeled and human comprehensible, as well as hidden layers used for computation that are trained and not observable during inference processes. Processing through each neuron adds the constituent input activity together (aggregates inputs) and passes it to output. In this way, generally, more input activity makes more output activity that propagates through the network. Outputs can connect to inputs strongly, weakly, or somewhere in between. This connection is rated with a normalized value between 0 and 1. As represented in the figure, a large dot corresponds to a large impact and vice versa.

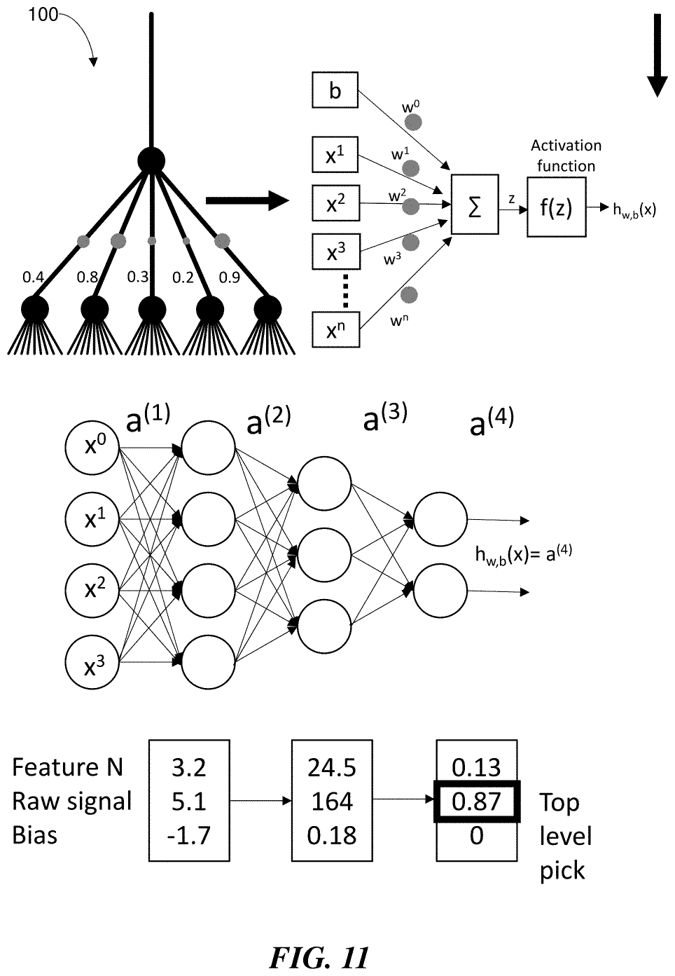

FIG. 11 depicts a block diagram an example embodiment procedure weighting relationship paths for an activation function used to determine outputs. Paths and/or nodes are weighted, with the weighting of each being altered as the system 100 learns from additional input over time. Activation function f(Z) may be represented by an aggregation of networked nodes (x) with weights (w) corresponding to the following equation:

z=Σᵢ₌₀ⁿWᵢxᵢ+b=W₀x₀+W₁x₁+ . . . +Wₙxₙ+b h_{w,b}(x)=a⁽²⁾=f(z)

Between first input and final output there may be layers of interconnections progressively refined according to equations:

z^{(l+1)}=W^{(l)}a^{(l)}+b^{(l)}

a^{(l+1)}=f(z^{(l+1)})

Where aᵢ^{(l)}: activation of unit i in layer 1, e.g. aᵢ⁽¹⁾=xᵢ=input data

a⁽²⁾=f(z⁽²⁾)=f(W⁽¹⁾a⁽¹⁾+b⁽¹⁾)

h_{W,b}(x)=a⁽⁴⁾=f(z⁽⁴⁾)=f(W⁽³⁾a⁽³⁾+b⁽³⁾)

In such an example subsystem 360 the final network layer may be used to represent categorical distribution. The system 100 employs a normalized exponential function. The result of the normalized function is a k-dimensional vector of real values between 0 and 1 that refines feature N raw signal bias into top level pick normalized 0-1:

$$P ( X = {x}_{i} ) = \frac{{e}^{z} k}{{\sum }_{n} {e}^{z} n} with z = f ( {x}_{i} ; W )$$

Using the weighted vectors to define connections, the subsystem 360 learns the relationships between the various inputs and corresponding outputs (or potential outputs). Thus, the constructed Neural Networks develop a numerical model based on these relationship (supervised learning). This approach is well known to be advantageous for modelling complex relationships, pattern recognition and data classification tasks given enough data. The system 100 continually supplies real time data, providing more than enough to compute useful results. Traditionally, expert domain knowledge would be required to derive an algorithm, and laborious and often inefficient and exceedingly variable results would be produced that often had to be recomputed for diverse patient or disorder sets. Here algorithm bias (b) is determined by expert/therapist 180 and individual features can be weighted by therapist 180 as part of the systematic refinement of algorithms and resultant data processing and iterative patient evaluation. All used signals can be weighted by the therapist 180 initially according to the impact. The weighting happens after the initial algorithm was trained on a larger crowd of people. If no weighting happens—the features are only weighted by the weights of the neural network. In case a signal/feature/etc. is weighted, the weights of the network additionally weight these signals but the impact on the output will be stronger. The weighting happens based on the initial patient assessment and the weighting of behavioral 137, circumstantial 136 and physiological signals 130.

Specific implementation of neural networks applicable for the example embodiment include long short-term memory neural networks, and more broadly, recurrent neural networks used for deep learning implementing feedback connections used for classifying, processing and making predictions based on time series data. Both architectures are able to capture states and transitions between states.

The multimodal machine learning subsystem 360, with its mixture of unsupervised learning, supervised learning, and non-learning modes, enables the behavior disorder management system 100 to learn a dynamic physiological profile for a user 110, to detect anomalies in said profile, to learn what physiological variations lead to said anomalies, and to classify physiological features into a predetermined set of classes. Baseline and ongoing physiological profiles of each user 110, of groups of users 110, and of the set of all users 110 are learned. By learning the dynamics and statistics of multiple physiological variables over time for each user 110 and for multiple users 110, a reliable mapping between physiological variables and behavioral disorders, and their severity, is determined for individual users 110 and for groups of users 110. The example embodiment is able to develop the physiological correlation or patterns that capture or describe the characteristics of psychiatric presentations that have physiological changes as part of their nature, i.e. agitated depression.

In operation, an example embodiment uses machine learning enabled by the subsystems (e.g., 360) described above to perform algorithm tailoring by first deriving an initial algorithm from stored data regarding a relevant population or subset of patients. This algorithm is adapted based on data e.g. that the patient feels uncomfortable: (1) when too many people are around, where input signals verify that this is based on a number of detectable wireless (e.g., Bluetooth® devices, and so receives weighting N times higher than other features; (2) Based on location where the closer in proximity the patient gets to home the stronger weight of this feature and/or bias are added to the neural network; (3) Based on time where panic tends to occur later in the day and so corresponds to stronger weight for this feature and/or bias added to the neural network; (4) Based on calendar entries where certain types of events trigger panic and so produces stronger weight for this feature and/or bias added to the neural network; (5) Based on foggy weather conditions where certain types of events trigger panic and so stronger weight is applied to this feature and/or bias added to the neural network. Additional adaptation of the algorithm is provided through processing therapist 180 insights, including monitored reaction to Zoe treatment that indicates (1) how a patient responds to the algorithm implementation and (2) what observations there are during the patient/arousal state. This is accomplished by weighing which features/inputs have a stronger impact to change a state in the patient.

From this processed data and adapted algorithm individual thresholds and correlations of patient data are determined, incorporating expert analysis and finetuning based upon therapist input and control of development. Weights for relationships between nodes are learned over time by incorporation of data including annotated panic events and patient experiences recorded in the data store and indexed for use. Weights are learned over time by identifying how physiology changes when applying a certain training technique of Zoe.

A combination of these weighted features determines the patient state with respect to presently manifested behavioral, circumstantial, and physiological characteristics observed and input into the system 100. In example embodiments certain features may be based on a threshold, e.g., a “distance from home” feature is only taken into account by the algorithm/system 100 when the observed distance exceeds a stored threshold. The relative weight of a feature may also depend on a threshold—e.g., the importance of a feature is directly proportional on another one. For example, when considering distance from home, a combination of time of day (threshold: no panic before 11:00 AM) computed with location (no panic within a radius of 1 mile around patient house—a feature only taken into account when radius larger than 1 mile) computed with hunger and computed with HRV.

The detection and classification signals may vary according to the disorder but a preferred embodiment for most disorders comprises a multi-level detection signal that indicates that a disorder event has been detected if the level is above a threshold. The level of the detection signal corresponds to the severity of the disorder event. The number of levels of the detection signal corresponds to the number of severity levels (e.g., in the previous example above there are four levels: normal, mild, moderate, and intense). In an alternative embodiment, the detection signal is a binary signal that indicates the presence or absence of a disorder event. This binary detection signal is based on the classification signals: if one or more non-normal classification signals are active, then the binary detection signal is active. The detection and classification signals are used to determine the number and severity of behavioral disorders for each user 110, for groups of users 110, and for all users 110. In an example embodiment, treatment effectiveness is based on the reduction in the number and severity of behavioral disorders compared to a baseline.

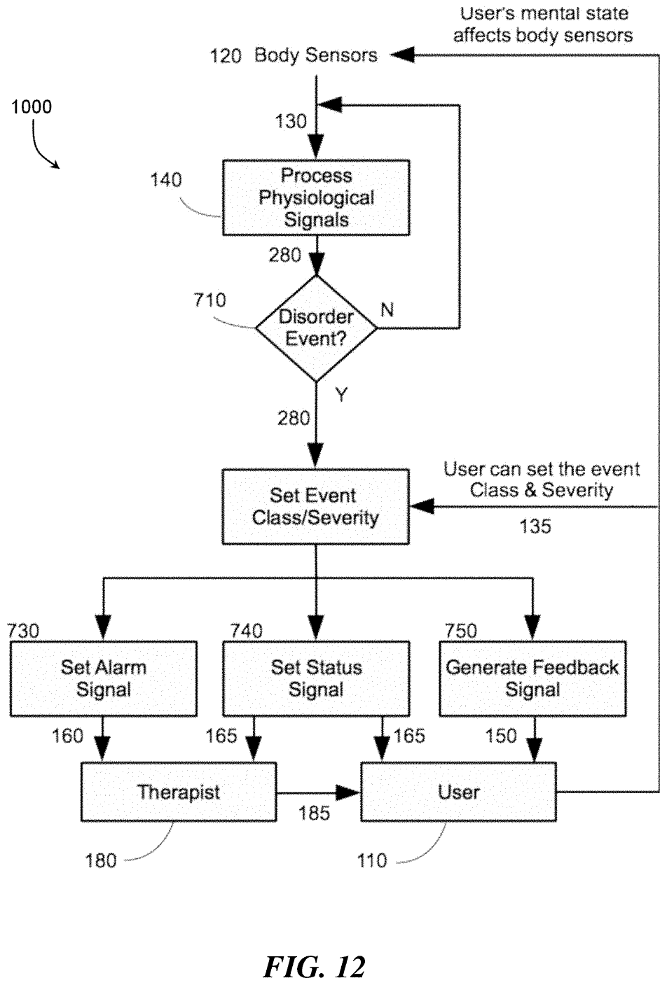

The signal flow is indicated in FIG. 7. Body sensors 120 transmits physiological signals 130 and other signals including those representing circumstantial 136 measures or behavioral 137 measures to the signal processing subsystem 210 of FIG. 6. The signal processing subsystem 210, or more specifically the machine learning subsystem 360 of FIGS. 6 and 9, generates a disorder event detection signal. The disorder event detection signal is based on the classification signals generated by the supervised learning subsystem 630 of the machine learning subsystem 360 of FIG. 9. In an example embodiment, if any of the non-normal disorder classes are active then the disorder event detection signal indicates that a disorder event has been detected. The level of the detection signal is the sum of the active non-normal events so that the level of the detection signal indicates the severity of the event. If the disorder event decision 710 is positive, then the event class and severity are set (FIG. 12). Signals from the user 110 via the status reporting device 170 can override the event class and severity. Once the event class and severity are set, the alarm signal 160 is set 730. The alarm signal 160 is the set point at which the user 110 is notified that a disorder event has been detected. and the status signals 165 are set 740 and the feedback signals are generated 750. The feedback physiological signals 150 are used to guide the user's 110 response. All this information processing is accomplished by the signal presentation subsystem 220 of FIG. 5. The alarm and status signals 165 are sent to the therapist 180 and the status and feedback signals are sent to the user 110. The therapist 180 provides guidance to the user 110 and this guidance helps the user 110 change the physiological variables that produced the disorder event.

In an example embodiment, the signal transformer 140 in FIG. 1 is located on a server. Communication between the body sensors 120, the signal transformer 140, and the therapist 180 is as follows. The body sensors 120 transmit physiological signals 130 to the status reporting device 170 via wireless protocol, such as Bluetooth®. The status reporting device 170 is wirelessly connected to a cloud network, such as the internet, via the cellular network and it transmits the physiological signals 130 to the signal transformer 140 via the internet. This embodiment is preferred when the status reporting device has limited computational capability and communication to the cellular network is reliable. An alternative embodiment in which the signal transformer 140 is located on the status reporting device 170 and communication to a cellular network is not required can be used if a status reporting device 170 of sufficient computational capability and storage is available, it is sufficiently portable, and it is convenient for the user 110 to view feedback signals. The status reporting device 170 may need to be periodically connected to an external storage device in this alternative embodiment to prevent loss of data.

The behavior disorder management system 100 described herein solves several problems with treating a variety of behavioral disorders. It permits treatment of the disorder in the user's 110 natural environment rather than in the confines of a therapist's 180 office. The system 100 not only detects and classifies disorder events based on physiological variables, it also provides feedback to the user 110 that can be used to control, lessen, or eliminate the disorder. The system 100 also provides a mechanism by which a therapist 180 can provide guidance and advice remotely to the user 110. Another feature of using this device for anxiety treatment is the virtual “constant contact” environment created by the capacity for communication between the user 110 and the therapist 180 which provides the user 110 with the important supportive knowledge that help is available. The effectiveness of the automated feedback as well as the clinician-provided feedback is used to personalize and improve the treatment as more disorder events are encountered. The inventive system 100 and method 1000 are helpful in the detection of anxiety disorders in hospital emergency rooms and will reduce the number of false positive cardiac events in which the underlying cause is anxiety disorder. The inventive system 100 and method 1000 also permit several clinicians representing several different disciplines to treat the user 110 simultaneously. The present invention solves many problems associated with previous systems and contains novel functionality not present in any existing device. Although the present invention has been described in considerable detail with reference to certain embodiments, other embodiments are possible. Therefore, the spirit and scope of the appended claims should not be limited to the description of the embodiments contained herein. This device 170 can be used either as a standalone device or in conjunction with therapist-assisted mode. The standalone version requires that the individual first record and transmit ECG recordings to the company for analysis and detection of the alert function particular to their physiological profile. Simultaneously, the user 110 will be training on the relaxation and biofeedback protocols, as described previously. Once a user 110 has developed the ability to voluntarily control heart physiological signal 130 to a set criterion specific to the user 110, the alert signal will be provided to the user 110 when the next disorder event takes place. Upon receiving this alert the user 110 will be guided to their previously trained relaxation procedure as well as be provided with the biofeedback display for that particular physiological signal 130.

Experimental Results

In a working implementation, 39 patients were equipped with a medical grade ECG device recording data over the course of multiple days. The measurements also include IMU data. The ECG sensors were placed on the chest and on the anterior right thigh. The data was captured and stored on the device and in the backend. All analysis and development were executed offline while using the data captured from the patients.

The methods used during the development of a panic detection alerting function cover data cleaning, denoising of the signal, and extracting time and frequency domain features of the heart rate components as recorded by the ECG. A variety of models have been used to test and predict the pre-panic, panic, and baseline states of a patient based on ECG signal. A prerequisite for this work was to manually annotate the states based on the patients and physicians' feedback. The severity of a panic related incident had to be classified by the data scientist on a scale from 1 to 10 to rate the severity of a panic event. Based on the IMU data, the movement state of a patient was determined as sleeping, moving or resting.

The ECG signal was cleaned and denoised to remove body movement, skin contact related issues, powerline interference and EMG noise. Body movement and skin contact related noise occurs in a frequency range between 1-10 Hz, whereas powerline induced noise is present at 60 Hz and electrical activity of muscles occurs between 50 and 150 Hz. Noise was removed using wavelet transforms and FIR (finite impulse response) filters, such as bandpass filters to reduce selected noise prone frequency components.

The recorded ECG uses a 1 kHz sampling frequency and the data was segmented into two-minute time windows with an overlap of 30%. The window size was consciously selected at two minutes to allow for a more robust extraction of non-linear HRV features and features in the time and frequency domain for each time window. The used features cover

Time domain:
RMSSD, MeanNN, SDNN, SDSD, CVNN, CVSD, MedianNN, MadNN, HCVNN, IQRNN, pNN50, pNN20, TINN, HTI

Frequency domain:
ULF, VLF, LF, HF, VHF, LnHF, LFn, HFn

Non-linear domain:
SD1, SD2, SD1SD2, CSI, CVI, CSI_Modified, GI, SI, AI, PI, SD1d, SD1a, C1d, C1a, SD2d, SD2a, ApEn, PAS, C2d, C2a, SDNNd, SDNNa, Cd, Ca, PIP, IALS, PSS, SampEn

Engineered features:
Relative change in time/frequency domain or non-linear features, IMU states

For the classification of the patient's states, a variety of models were evaluated to select the best model for classifying the overlapping time windows in baseline, panic and pre-panic states. The selected and implemented model is XGBoost. Insights into the model performance and feature importance were provided using the SHAP library to determine the importance of each feature.
Use models cover: XGBoost, LightGBM, SVC, MLP, Random Forest, Gradient boosting classifier, GaussianNB

The classification was carried out for each individual subject and the model performance was evaluated on a per patient basis. The results show:
Option 1: Data for 9 patients was of sufficient quality to use it for panic/patient state classification. 82.2% of panic attacks (pre-panic and panic) were correctly detected, whereas 18.8% of panic attacks were missed. Non-panic events, meaning baseline, were wrongly classified 40 times, resulting in 1 out of 3 panic detections being a false alarm.
Option 2: 69 of 85 recorded panic attacks were correctly classified, whereas 16 of 85 time attacks were wrongly classified, meaning they were missed. Non-panic events, meaning baseline, were wrongly classified 40 times, resulting in 1 out of 3 panic detections being a false alarm.

As is made evident by the above example and the disclosure herein, the present invention provides a system for detecting, managing, and potentially mitigating disorders including but not limited to anxiety and panic attacks. In the present invention, these physiological personality profiles present themselves without needing any self-report or psychological inquiry. This physiologic taxonomy emerges from the monitoring, assessing, analyzing, and patterning of these variables conjoined with the inventive system for understanding these relationships. These profiles are then synthesized into a psychological condition database that is used to more efficiently diagnose and treat these disorders. In the case of panic, profiles of this nature streamline the diagnosis of panic thus reducing unnecessary patient suffering and costly testing.

As a further example specific to panic attacks, traditional psychotherapeutic interventions do not address panic disorder's extreme activation of the limbic system causing physiological arousal. This conditioned response (fight or flight) makes cognitive behavioral intervention difficult to access. The present invention enables early detection by measuring ECG and HRV values before the limbic system is activated and therefore allows the patient to implement prior training in both relaxation and biofeedback to control the arousal which produces the panic attack. The control of the body's physiological response is central to the operating paradigm of the present invention. That is, the present invention is capable of recognizing indications of the onset of anxiety or panic in the process before the onset actually occurs, thereby enabling the mitigation or prevention of the onset of anxiety or panic. This process is replicable with other disorders as well by similarly determining a chain of actions that lead to the expression of the disorder (such as the panic attack or other disorder), identifying and measuring biological characteristics of the patient that occur earlier in the chain of actions, and when a threshold measurement is sensed, intervening and taking action using the system of the present invention to detect and output classification and recommended action to reduce or fully avert the full expression of the disorder.

To any extent utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about” and “approximately” are intended to cover variations that may existing in the upper and lower limits of the ranges of subjective or objective values, such as variations in properties, parameters, sizes, and dimensions. In one non-limiting example, the terms “about” and “approximately” mean at, or plus 10 percent or less, or minus 10 percent or less. In one non-limiting example, the terms “about” and “approximately” mean sufficiently close to be deemed by one of skill in the art in the relevant field to be included. As utilized herein, the term “substantially” refers to the complete or nearly complete extent or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A system for detecting and managing behavioral disorders of a user, the system comprising:
one or more computing devices comprising a signal processing subsystem and a signal presentation subsystem;
one or more wearable body sensors that measure one or more physiological indicator variables to obtain physiological data comprising heart rate variability data and transmit the physiological data over a communication network to the signal processing subsystem, wherein the signal processing subsystem determines onset of a panic attack or other psychological disorder of the user based on the physiological indicator variables and the physiological data and derives an algorithm from differences between a baseline and a shift in physiological arousal exceeding predetermined threshold limits;
a status reporting device connected to the communication network that receives feedback signals from the signal presentation subsystem; and
an instruction module connected to the communication network;
wherein the signal processing subsystem uses the algorithm and machine learning to perform detection and classification, and outputs detection and classification signals to the signal presentation subsystem, and both are connected to the communication network.

2. The system for detecting and managing behavioral disorders according to claim 1, wherein the signal processing subsystem comprises a remote computing device that transmits physiological data over the communication network to the signal presentation subsystem comprising a second remote computing device and the signal presentation subsystem transmits feedback signals to the status reporting device connected to the communication network and the instruction module connected to the communication network.

3. The system for detecting and managing behavioral disorders according to claim 1, wherein the status reporting device is connected to the communication network via a Wi-Fi network.

4. The system for detecting and managing behavioral disorders according to claim 1, further comprising alarm and status signals that are sent to a therapist who communicates remotely with the user.

5. The system for detecting and managing behavioral disorders according to claim 1, wherein physiological indicators are used in combination to detect an onset of a panic attack or other behavioral disorder or create an algorithm for detection and the physiological indicators comprise two or more of: heart rate; heart rate variability; electrocardiogram (ECG); respiratory rate; galvanic skin response; electromyography (EMG); electrooculography (EOG); electroencephalography—Fast Fourier transform analysis (EEG-FFT); skin temperature; posture; and acceleration.

6. The system for detecting and managing behavioral disorders according to claim 5, wherein onset indication functions in combination with real time medical/symptomatic treatment, wherein the real time medical/symptomatic treatment comprises psychological concepts of one or more of conditioning and learning, baseline, degrees of freedom, double helix, and a nature vs. nurture continuum independently or in conjunction with progressive muscle relaxation, guided imagery, or other relaxation techniques.

7. The system for detecting and managing behavioral disorders according to claim 1, further comprising treatment using psychological concepts of one or more of conditioning and learning, baseline, degrees of freedom, double helix, and a nature vs. nurture continuum independently or in conjunction with progressive muscle relaxation, guided imagery, or other relaxation techniques.

8. The system for detecting and managing behavioral disorders according to claim 1, wherein the signal processing subsystem comprises a machine learning subsystem that uses machine learning to map features representing physiological, behavioral and circumstantial data over time into reliable behavioral disorder detection and classification signals.

9. The system for detecting and managing behavioral disorders according to claim 8, wherein the machine learning subsystem comprises unsupervised learning and supervised learning modes, and wherein the machine learning subsystem unsupervised learning mode comprises one or more of clustering and autoencoding to learn higher-order features.

10. The system for detecting and managing behavioral disorders according to claim 8, wherein the machine learning subsystem comprises one or more of a support vector machine, a neural network, a statistical learning algorithm, or a combination thereof.

11. The system for detecting and managing behavioral disorders according to claim 1, wherein the signal processing subsystem comprises a preprocessing subsystem, a feature extraction subsystem, and a machine learning subsystem.

12. The system for detecting and managing behavioral disorders according to claim 11, wherein the preprocessing subsystem normalizes physiological signals for each time frame by (a) computing a mean-subtracted and standard deviation normalized signal, (b) computing a range normalized signal, producing a signal range from about 0 to 1 or from −1 to 1, or (c) by computing a ratio of the signal to its L1 or L2 norm.

13. The system for detecting and managing behavioral disorders according to claim 11, wherein the feature extraction subsystem extracts a set of features by determining a closest match of the signal over each time frame to a dictionary of functions and/or a predefined set of functions.

14. The system for detecting and managing behavioral disorders according to claim 11, wherein the feature extraction subsystem computes one or more of a Fast Fourier Transform, a wavelet transform, a principal components analysis, an independent components analysis, or a bank of bandpass filters.

15. The system for detecting and managing behavioral disorders according to claim 1, wherein the system monitors automated feedback and characterizes the automated feedback for effectiveness.

16. The system for detecting and managing behavioral disorders according to claim 1, wherein clinician-provided feedback is monitored and characterized for effectiveness, and wherein machine learning is used to improve treatment effectiveness based on most effective automated and clinician-provided feedback.

17. The system for detecting and managing behavioral disorders according to claim 16, wherein treatment effectiveness is estimated using a reduction in a number or severity of behavioral disorders from a baseline.

18. The system for detecting and managing behavioral disorders according to claim 1, wherein data are collected from multiple users and used to establish the baseline and a baseline physiological profile for each user and/or for a group of users.

19. The system for detecting and managing behavioral disorders according to claim 18, wherein the baseline and the baseline physiological profile are based on classification and detection signals.

20. The system for detecting and managing behavioral disorders according to claim 1, wherein the baseline comprises heart rate variability data values derived from clinical data sets or aggregated data sets collected from multiple users.

21. The system for detecting and managing behavioral disorders according to claim 1, wherein the processing subsystem uses machine learning to adjust an algorithm according to physiological indicators that comprise two or more of: heart rate; heart rate variability; electrocardiogram (ECG); respiratory rate; galvanic skin response; electromyography (EMG); electrooculography (EOG); electroencephalography—Fast Fourier transform analysis (EEG-FFT); skin temperature; posture; and acceleration based feedback of the user or specific physiology of the user in order to modify treatment.

22. The system for detecting and managing behavioral disorders according to claim 1, wherein the system instructs the user in real-time, using the instruction module, with management techniques comprising biofeedback and/or neurofeedback for a detected psychological disorder, wherein feedback signals and status signals are sent to the user via the status reporting device comprising one of: a smart watch, a smartphone, a tablet computer, a laptop, or personal computing device presenting one or more of visual, audio, textual, vibrational or kinesthetic signals to the user.

23. The system for detecting and managing behavioral disorders according to claim 1, further comprising the processing subsystem using a machine learning subsystem to identify user psychological patterns and/or physiological patterns demonstrating characteristics of psychiatric presentations that comprise physiological changes, enabling a physiological signal to a set criterion specific to the user, triggering an alert signal that will be provided to the user when a next disorder event takes place based on user psychological patterns and/or physiological patterns.

24. The system for detecting and managing behavioral disorders according to claim 1, wherein the behavioral disorders of the user comprise one or more of panic disorder, anxiety or depression of the user.

25. The system for detecting and managing behavioral disorders according to claim 1, wherein the one or more wearable body sensors comprise electrodes or charge and/or voltage measuring devices configured to record ECG data from the user.

26. The system for detecting and managing behavioral disorders according to claim 1, wherein the baseline comprises heart rate variability data values determined using a machine learning subsystem to analyze prior recorded heart rate variability of one or more users previously transmitted by one or more wearable body sensors attached to the one or more users.

27. The system for detecting and managing behavioral disorders according to claim 1, wherein when heart rate variability reduces by a predetermined percentage over a predetermined time, the predetermined percentage and predetermined time being specific to the user based on user psychological patterns and/or physiological patterns, an alert signal is provided to the user indicating a disorder event is occurring.

28. A system for detecting and managing behavioral disorders of a user, the system comprising:
one or more wearable body sensors that measure one or more physiological indicator variables comprising heart rate variability to obtain physiological data and transmit physiological data to a signal processing subsystem comprising a computing device;
wherein the signal processing subsystem determines onset of a panic attack or other psychological disorder based on the physiological indicator variables and the physiological data to derive an algorithm from differences between a baseline and a shift in physiological arousal exceeding predetermined threshold limits;
a signal presentation subsystem comprising a computing device that electronically transmits feedback signals to a status reporting device;
an instruction module; and
wherein the signal processing subsystem uses the algorithm and machine learning to perform detection and classification, and transmits detection and classification signals to the signal presentation subsystem, and the signal processing subsystem, the signal presentation subsystem, the instruction module and the status reporting device are in electronic communication.

29. The system for detecting and managing behavioral disorders according to claim 28, wherein the signal processing subsystem and the signal presentation subsystem are both located on the status reporting device and no connection to an external communication network is required.

30. A method for detecting and managing behavioral disorders of a user, the method comprising:
one or more wearable body sensors measuring one or more physiological indicator variables comprising heart rate variability to obtain physiological data from the user wearing the one or more wearable body sensors;
the one or more wearable body sensors wirelessly transmitting the physiological data over a communication network to a signal processing subsystem comprising a remote computing device;
the signal processing subsystem determining onset of a panic attack or other psychological disorder based on the physiological indicator variables and the physiological data and deriving an algorithm from differences between a baseline and a shift in physiological arousal exceeding predetermined threshold limits;
the signal processing subsystem performing detection and classification using the algorithm and machine learning, and transmitting detection and classification signals to a signal presentation subsystem, comprising the remote computing device, that transmits feedback signals to a status reporting device presenting feedback signals and status signals to the user; and
an instruction module presenting to the user appropriate steps for management and treatment of a detected psychological disorder;
wherein the signal processing subsystem, the signal presentation subsystem, the instruction module and the status reporting device are connected to the communication network.
